(19) United States
(12) Patent Application Publication
(10) Pub. No.: US 2008/0221533 A1
(43) Pub. Date: Sep. 11, 2008

(54) IMPLANTABLE DEVICE WITH BIOABSORBABLE LAYER, KIT AND METHOD FOR USE THEREWITH, AND APPARATUS FOR PREPARING SAME

(75) Inventor: Debra Anne MATITYAHU, Los Altos, CA (US)

Correspondence Address: Dorsey & Whitney LLP;US Bank Center, 1420 Fifth Avenue, Suite 3400, Seattle, WA, 98101-4010, US

(73) Assignee: ANTHEM ORTHOPAEDICS LLC, Los Altos, CA (US)

(21) Appl. No.: 12/044,961

(22) Filed: Mar. 8, 2008

Related U.S. Application Data
(60) Provisional application No. 60/905,942, filed on Mar. 9, 2007.

(51) Int. Cl. A61M 35/00; A44C 7/00

(52) U.S. Cl. 604/290; 63/12; 604/289; 606/188

(57) ABSTRACT

An implantable device for use in an externally-accessible pierced opening in a mammalian body is disclosed. The implantable device has an elongated member adapted for insertion into the opening. A bioabsorbable material is provided on at least a portion of the elongated member and a pharmaceutical agent is carried by the bioabsorbable material for eluting into the mammalian body when the elongated member is disposed in the opening. An apparatus for use with a supply of bioabsorbable material to prepare an implantable device for use in an externally-accessible pierced opening in a mammalian body and a method of delivering a medicament to an externally-accessible pierced opening in a mammalian body are also disclosed. A kit including a container having a bioabsorbable material and medicament for applying to the implantable device prior to insertion into the externally-accessible pierced opening is also disclosed.

### RELATED APPLICATION DATA

This application claims benefit of provisional application Ser. No. 60/905,942, filed Mar. 9, 2007, the entire content of which is expressly incorporated by reference herein.

### FIELD OF THE INVENTION

The invention relates generally to implantable body piercing devices and more particularly to implantable body-piercing devices having medicaments.

### BACKGROUND

Typically, after having an ear pierced, the individual is required to wear a surgical stainless steel earring for a period of at least six to eight weeks in order to allow the piercing to heal. During this time, the individual is responsible for keeping the piercing clean and free from debris in order to minimize infection and to promote healing by, typically, applying hydrogen peroxide or alcohol with a cotton ball, cotton swab, or gauze to the pierced region. Although this can be an effective method to treat the pierced area, it requires careful and tedious care of an area that may be cumbersome or difficult to treat because of poor visibility of the affected region or awkward handling of moistened swabs and gauze.

Currently available delivery systems for body piercing therapeutics are limited. One such delivery system includes drops, which carry issues of expense, inconvenience, patient noncompliance by overuse, underuse, or inappropriate frequency of use, as well as difficulty in delivery of medications by certain patients. Other delivery systems include injections and bulky implants placed within the earlobe that can be filled with material that is extruded. Regardless of the foregoing method chosen, there is often a significant chance that when the body part is pierced, infection will occur using these present methods.

In view of the foregoing, there is a need in the art apparatus and methods for applying or affixing a medicament or pharmaceutical agent or agents onto an implantable body piercing device to aid in the healing process.

### SUMMARY OF THE INVENTION

The invention is generally directed to an implantable device for use in an externally-accessible pierced opening in a mammalian body. The implantable device has an elongated member adapted for insertion into the opening. A bioabsorbable material is provided on at least a portion of the elongated member and a pharmaceutical agent is carried by the bioabsorbable material for eluting into the mammalian body when the elongated member is disposed in the opening.

An apparatus can also be provided for use with a supply of bioabsorbable material to prepare an implantable device for use in an externally-accessible pierced opening in a mammalian body. The apparatus may include a support for temporarily securing to the implantable device and an application device for applying the bioabsorbable material to the implantable device. The application device may be arranged to contact the implantable device secured by the support with the supply of bioabsorbable material and pharmaceutical agent. The application device may also be capable of applying the bioabsorbable material and pharmaceutical agent to the implantable device.

A kit can also be provided which includes a package having an implantable device for implanting into an externally-accessible pierced opening in a mammalian body. The implantable device includes an implantable portion. A container may also be included in the kit having a bioabsorbable material and medicament for applying to the implantable device prior to insertion into the externally-accessible pierced opening.

The invention is also directed to a method of delivering a medicament to an externally-accessible pierced opening in a mammalian body. The method generally includes the steps of applying a bioabsorbable material and a medicament to an implantable device, implanting the implantable device into the externally-accessible pierced opening, and eluting the medicament.

Other features of the present invention will become apparent from the following description along with the accompanying drawings.

### BRIEF DESCRIPTION OF THE DRAWINGS

FIG. 1a is an isometric view of an exemplary embodiment of an implantable device for use in an externally-accessible pierced opening in a mammalian body with a bioabsorbable pharmaceutical feature and a typical clasp.

FIG. 1b is an isometric view of an exemplary embodiment of an implantable device for use in an externally-accessible pierced opening in a mammalian body t with a bioabsorbable pharmaceutical feature shown with the clasp fitted to the post.

FIG. 1c is a front view of an exemplary embodiment of an implantable device for use in an externally-accessible pierced opening in a mammalian body with a bioabsorbable pharmaceutical feature and a typical clasp.

FIG. 1d is a sectional side view taken along the line 1d-1d of FIG. 1c of an exemplary embodiment of an implantable device for use in an externally-accessible pierced opening in a mammalian body identifying the bioabsorbable pharmaceutical feature and a typical clasp.

FIG. 2 is a diagrammatic example of a process for creating a coating on an implantable device for use in an externally-accessible pierced opening in a mammalian body by a spray method using a variety of pressurized sources containing liquid bioabsorbable medium.

FIG. 3 is a diagrammatic example of another embodiment of a process for creating a coating on an implantable device for use in an externally-accessible pierced opening in a mammalian body using a spray method based on a syringe-like vessel that is pressurized by a variety of actuators acting on a plunger.

FIG. 4a is a front view of a stand-alone system for applying a spray coating, where a syringe-like vessel contains the bioabsorbable liquid medium and is fitted into a carrier and an implantable device for use in an externally-accessible pierced opening in a mammalian body is fitted into a rotary shaft.

FIG. 4b is a front view of the stand-alone system for applying a spray coating of FIG. 4a, where the syringe-like vessel is fitted into a carrier and engaged with the actuator.

FIG. 4c is a front view of a stand-alone system for applying a spray coating of FIG. 4a, where the actuator applies a force against the plunger of the syringe-like vessel and causes the liquid bioabsorbable medium to spray from the nozzle onto the implant while it rotates.

FIG. 4d is a front view of a stand alone system for applying a spray coating of FIG. 4a, where the actuator retracts slightly to reduce the pressure in the syringe-like vessel and the implantable device for use in an externally-accessible pierced opening in a mammalian body is heated to aid in drying the coating.

FIG. 5 is a front view of an exemplary drive system for the rotary shaft of the stand-alone system for applying a spray coating of FIG. 4a showing a direct drive connection with a motor.

FIG. 6 is a front view of another embodiment of an exemplary drive system of the stand-alone system for applying a spray coating of FIG. 4a for the rotary shaft showing a belt or gear drive connection to a motor.

FIG. 7 is a front view of another embodiment of an exemplary stand-alone system for applying a spray coating where multiple syringes-like vessels as shown in FIG. 4a can be used to apply coatings with different compositions.

FIG. 8 is a front view of an exemplary embodiment of two syringe-like vessels of the stand-alone system for applying a spray coating of FIG. 7 showing two plungers tied together with a cross-bar for uniform delivery of each component.

FIG. 9 is a front view of a stand-alone system for applying a spray coating of FIG. 4a oriented in a horizontal position, where a tray collects drips or leaks from the nozzle.

FIG. 10 is an isometric view of a tray with a single round cup containing liquid bioabsorbable medium for use in an embodiment of a process for creating a coating on an implantable device for use in an externally-accessible pierced opening in a mammalian body.

FIG. 11 is an isometric view of a tray with multiple round cups containing liquid bioabsorbable medium of FIG. 10 for use in another embodiment of a process for creating a coating on an implantable device for use in an externally-accessible pierced opening in a mammalian body.

FIG. 12 is an isometric view of a tray with a single rectangular cup containing liquid bioabsorbable medium for use in another embodiment of a process for creating a coating on an implantable device for use in an externally-accessible pierced opening in a mammalian body.

FIG. 13 is an isometric view of a tray with multiple rectangular cups containing liquid bioabsorbable medium of FIG. 12 for use in an embodiment of a process for creating a coating on an implantable device for use in an externally-accessible pierced opening in a mammalian body.

FIG. 14a is a front view of a mechanism for performing a an embodiment of a process for creating a coating on an implantable device for use in an externally-accessible pierced opening in a mammalian body including a dipping process to coat an implantable device of FIG. 1a for use in an externally-accessible pierced opening in a mammalian body, whereby a tray with receptacles containing liquid bioabsorbable medium of FIG. 13 can be placed within a carrier.

FIG. 14b is a front view of the mechanism of FIG. 14a for performing a dipping process to coat an implantable device for use in an externally-accessible pierced opening in a mammalian body, whereby a tray with receptacles containing liquid bioabsorbable medium is shown mounted within a carrier

FIG. 14c is a front view of the mechanism of FIG. 14a for performing a dipping process to coat an implantable device for use in an externally-accessible pierced opening in a mammalian body, where the implant is dipped in receptacle ‘A’.

FIG. 14d is a front view of the mechanism of FIG. 14a for performing a dipping process to coat an implantable device for use in an externally-accessible pierced opening in a mammalian body, where the dipped coating is dried with heater elements and the tray is indexed.

FIG. 15a is a front view of an exemplary embodiment of a holder for accommodating the post feature of an implantable device for use in an externally-accessible pierced opening in a mammalian body of FIG. 1.

FIG. 15b is a front view of the holder of FIG. 15a with the post feature of an implantable device for use in an externally-accessible pierced opening in a mammalian body mounted.

FIG. 16 is a front view of the holder of FIG. 15a with an implantable device for use in an externally-accessible pierced opening in a mammalian body mounted thereon as shown in FIG. 15b and under a spray nozzle.

FIG. 17 is a front view of another embodiment of a mechanism for performing a dipping process to coat an implantable device for use in an externally-accessible pierced opening in a mammalian body, where the holder of FIG. 15a is fitted to the post of the implant.

FIG. 18a is front sectional view of the implantable device of FIG. 1 a prior to being placed within a molding cup, taken along the line 18a-18a of FIG. 18b.

FIG. 18b is a top view of the implantable device of FIG. 1a, prior to being placed within a molding cup.

FIG. 18c is a front view of the implantable device of FIG. 1a, prior to being placed within a molding cup.

FIG. 19a is front sectional view of the implantable device of FIG. 1a, after being placed within a molding cup, taken along the line 19a-19a of FIG. 19b.

FIG. 19b is a top view of the implantable device of FIG. 1a, after being placed within a molding cup.

FIG. 19c is a front view of the implantable device of FIG. 1a, after being placed within a molding cup.

FIG. 20a is a front view of another embodiment of a process for creating a coating on an implantable device for use in an externally-accessible pierced opening in a mammalian body including a lever mechanism with liquid bioabsorbable medium contained within a pressurized can to apply a coating to the implantable device.

FIG. 20b is a front view of the process including a lever mechanism with liquid bioabsorbable medium contained within a pressurized can of FIG. 20a, where a coating is applied to an implantable device for use in an externally-accessible pierced opening in a mammalian body.

FIG. 21a is a front view showing implantable devices for use in an externally-accessible pierced opening in a mammalian body with slits and a hollow core.

FIG. 21b is a side section view of the implantable device of FIG. 21a taken along the line 21b-21b of FIG. 21a, showing implantable devices for use in an externally-accessible pierced opening in a mammalian body with slits and a hollow core.

FIG. 21c is a side section view, similar to FIG. 21b, showing another embodiment of an implantable device for use in an externally-accessible pierced opening in a mammalian body with slits and a hollow core.

FIG. 22a is a front view showing implantable devices for use in an externally-accessible pierced opening in a mammalian body with holes and a hollow core.

FIG. 22b is a side section view of the implantable device of FIG. 22a taken along the line 22b-22b of FIG. 22a, showing implantable devices for use in an externally-accessible pierced opening in a mammalian body with holes and a hollow core.

FIG. 22c is a side section view, similar to FIG. 22b, showing another embodiment of an implantable device for use in an externally-accessible pierced opening in a mammalian body with holes and a hollow core.

FIG. 23a is an isometric view of another embodiment of an implantable device for use in an externally-accessible pierced opening in a mammalian body with a helical groove along the post feature.

FIG. 23b is an isometric view of another embodiment of an implantable device for use in an externally-accessible pierced opening in a mammalian body with longitudinal ribs or ridges along the length of the post feature.

FIG. 23c is an isometric view of another embodiment of an implantable device for use in an externally-accessible pierced opening in a mammalian body with circumferential ribs or ridges along the length of the post feature.

FIG. 23d is an isometric view of another embodiment of an implantable device for use in an externally-accessible pierced opening in a mammalian body with dimples, pockets, or scallops, or combinations thereof on the surface of the post feature.

FIG. 23e is an isometric view of another embodiment of an implantable device for use in an externally-accessible pierced opening in a mammalian body with an undercut or reduced diameter section along the length of the post feature.

FIG. 24a is an isometric view of another embodiment of an implantable device for use in an externally-accessible pierced opening in a mammalian body with a pre-fabricated sleeve before placement on the post feature.

FIG. 24b is an isometric view of the implantable device of FIG. 24a showing the pre-fabricated sleeve placed upon the post feature.

FIG. 25a is a perspective view showing an exemplary diagrammatic view of the method of insertion of the implantable device of FIG. 1a into an externally-accessible pierced opening in a mammalian body, showing the human head from behind the left ear and including a piercing in the ear lobe.

FIG. 25b is a perspective view of the method illustrated in FIG. 25a, showing an isolated view of the left ear with the implantable device placed within the piercing in the ear.

FIG. 25c is a cross sectional view, taken along the line 25c-25c of FIG. 25b, showing the tissue relative to the implantable device of FIG. 1a.

FIG. 26 is a view of an exemplary embodiment of a kit containing an implantable device for use in an externally-accessible pierced opening in a mammalian body and a molding cup of FIG. 18c.

### DETAILED DESCRIPTION OF DRAWINGS

As described herein, an implantable device for use in an externally-accessible pierced opening in a mammalian body is provided. More specifically, the implantable device is for use as a body piercing and has attached, coated, adhered or applied thereto a pharmaceutical agent in a sustained release medium. The implantable device may be a body piercing implant acceptable for use in any body part, including any type of device used for body piercing in any location of the mammalian body. Common body piercings include, but are not limited to, ears, nose, tongue, and umbilicus. For purposes of simplicity and understanding, otologic implants, such as earrings, pins, pegs, or posts, are described herein. However, the disclosure may be equally applied to any body piercing device.

The implantable device generally has a body including an elongated member with an implantable portion. The implantable portion of the elongated member is positioned and sized to be received within the externally-accessible pierced opening and has a bioabsorbable material on a surface thereof which carries a medicament and is adapted for eluting the medicament. While specific implantable devices are described and illustrated herein, variations thereon and equivalent devices acceptable for the purposes provided would not depart from the overall scope of the present invention.

According to an embodiment of the implantable body piercing device, the implant is capable of releasing a medicament or other substance, including for instance, pharmaceutical agent or agents. More particularly, a bioabsorbable coating(s), sleeve or other feature may be applied or affixed to the implant containing a pharmaceutical agent or agents for the purpose of eluting a medicament during the healing process when the implant is disposed in the pierced opening, which may help to minimize the risk of infection or mitigate pain, and combinations thereof. The implant may be fabricated in any of a number of different ways as will be described herein, but in one embodiment, may include one or more layers, at least one of which degrades, erodes or otherwise diminishes and becomes bioavailable over time, to release at least one layer having one or more pharmaceutical agents.

The present invention as disclosed herein is an implantable device for use in an externally-accessible pierced opening in a mammalian body, and includes an otologic implant, for example an earring, stud, pin, or post, having an outer surface which has a bioabsorbable coating(s), cover, or sleeve that elutes a therapeutic compound within a body piercing or the pierced opening, such as an ear piercing, for a prescribed period of time.

The bioabsorbable coating(s), cover or sleeve can be made from any suitable bioabsorbable material or combination of materials. Acceptable materials may include hydrophobic or hydrophilic polysaccharides or any suitable material that is biocompatible and bioabsorbable. Materials can also include poly ester amide (PEA) type polymers, hydrogels, polylactic acid (PLA), polyglycolic acid (PGA), or combinations thereof to form co-polymers of PLA and PGA, also know as poly(lactide-co-glycolide). The medicament or pharmaceutical agent may be provided in any suitable concentration for the applicable treatment. The medicament or pharmaceutical agent can be dispersed in a sustained release medium material that is coated over a surface of the body piercing device or implantable device or portion thereof. Alternatively, the medicament can be impregnated into a space within the implantable portion or a combination of the foregoing. Preferably, the bioabsorbable material can be impregnated with a pharmaceutical agent, such as but not limited to an analgesic, anesthetic, anti-inflammatory, steroid, antiglaucomatous, or other medicament or combination of any of the foregoing, which is carried by the material and is eluted over a period of time, for example over a one to 45 day period, and more preferably, a period of approximately six (6) weeks. An eluting agent, chemical, or drug may be preferably impregnated into the material of the coating, and may be impregnated in any suitable manner, but can also be mixed in any suitable manner with the material of the bioabsorbable medium, including but not limited to, as microparticles or macromolecules incorporated into the matrix of the bioabsorbable material. Any elution rate suitable for the particular treatment may be used for the present invention. Drug release kinetics and coating degradation times can be tailored to meet the specifications of the specific drug and its efficacy on the target tissue. As one non-limiting example, the analgesic concentration can be sufficient to allow for clinically relevant pain relief that is maintained during the elusion phase.

One medium type acceptable for use with an embodiment of the invention includes, for instance, a medium having polysaccharide type materials which can be degraded with enzyme mediated digestion, whereby the erosion process begins from the outermost tissue contacting surface and propagates inward. This type of degradation process preserves the integrity of the coating closest to the otologic implant and should prevent premature loss of coating pieces and fragments.

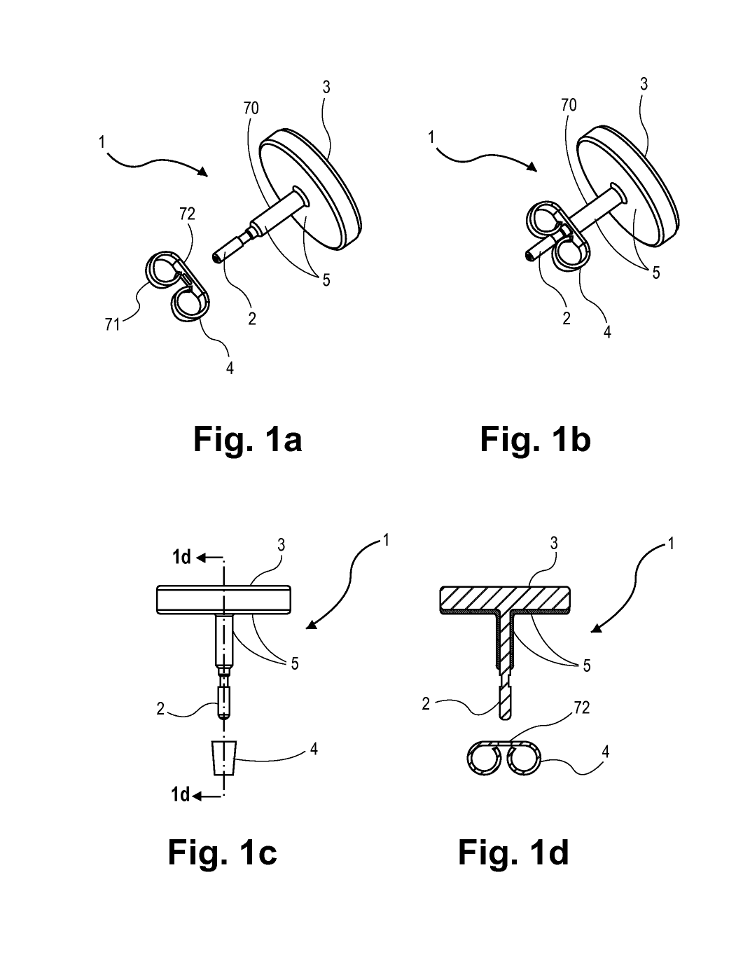

As depicted in the drawings, FIG. 1a shows a first embodiment of an otologic implant with a bioabsorbable feature 1 consisting of a body 70 having an elongate member or post feature 2, a terminal feature 3, a clasp element 4, and a bioabsorbable feature 5. In this exemplary embodiment, the post feature 2 can be any elongated member, with a circular, oval, octagonal, or any other cross-sectional geometry and can have a uniform cross-section along its length or can be inwardly or outwardly tapered. Additionally, the post feature 2 may be substantially straight or curved or be a portion of a substantially round hoop. The post feature 2 is intended to fit within the piercing made in any region of the ear, including but not limited to, the lobe or any cartilaginous regions of the ear. The post feature 2 may include an implantable portion for implanting into the ear. The terminal feature or member or element 3 overlies the pierced opening and prevents the end of the post feature 2 from pulling through the piercing, and merely has to be substantially large enough to prevent its passage through the piercing during normal use.

The terminal feature 3 can be a ball, a flange, as depicted in FIG. 1a, or any other shape, including decorative, symbolic, or ornamental features, given that they are large enough to prevent the otologic implant from unintentionally passing through the piercing in the ear during normal use. The terminal feature preferably overlies the hole in the mammalian body, and more preferably has a sufficient diameter or other transverse dimension so as to completely cover the hole. In the illustrated embodiment, the terminal feature has an outer or front surface 71 and an inner or back surface 72 for engaging the mammalian body. The post feature 2 extends from the inner surface 72 and preferably extends perpendicular to the inner surface 72. The clasp element 4 can have spring-like features, as shown in FIG. 1a, that are known in the art, to provide secure fixation to the post feature 2, either with (as depicted) or without a recessed groove on the post feature 2 for engagement with the spring-like features of the clasp element 4. The clasp element 4 can be of any style known in the art, either based purely on a friction fit, by means of a spring-feature or elastomer, or by engaging mechanically with a groove, ridge, barb or any other element on the post feature 2 (not shown). When a clasp element 4 is provided, the post 2 may preferably have a free end for receiving the clasp. A clasp feature 4 may not be necessary if the post feature 2 is substantially curved such that it hooks or encircles the tissue of the piercing.

The bioabsorbable feature 5 is formed of the bioabsorbable medium including medicament or pharmaceutical agent and can be affixed to both the post feature 2 and terminal feature 3, as depicted in FIG. 1 a, or isolated to either the post feature 2 or terminal feature 3 (not shown). To this end, the bioabsorbable material may be disposed on at least a portion of the back surface of the terminal element so as to overlie the pierced opening. Additionally, a bioabsorbable feature 5 can be affixed to the clasp element 4 (not shown), in order to provide additional medicament to the pierced region. The clasp element 4 is shown in FIG. 1b mounted onto the post feature 2, as is understood by typical users. The bioabsorbable feature 5 can be localized to just a portion of the post feature 2, so as not to interfere (or cause an interference fit) with the placement of the clasp element 4, as depicted in FIGS. 1a-1c. Alternatively, the bioabsorbable feature 5 material can extend the entire length of the post feature 2 (not shown), such that the bore of the clasp element 4, either fits over any additional thickness from the bioabsorbable feature 5 or causes the bioabsorbable feature 5 to collapse like a bellows, if the material is suitably compliant, or the material from the bioabsorbable feature 5 is allowed to be mechanically displaced or removed at the tip of the post feature 2 because of an interference fit with the clasp element 4. The section view depicted in FIG. 1d shows an example of regions of the post feature 2 and the inner surface 72 of the terminal feature 3 that are affixed with the bioabsorbable feature 5.

The otologic implant with a bioabsorbable feature 1 depicted in FIGS. 1a-1d can have a post feature 2 with a nominal diameter ranging between 0.001″ and 1.000″, or more specifically between 0.020″ and 0.125″, or even more narrowly between 0.030″ and 0.0625″. Additionally, the post feature 2 can be hollow in order to reduce the mass of material given a specified outer diameter or to be purely decorative, as in the case of lobe tunnels used for stretched lobe piercings. The length of the post feature 2 can range between 0.050″ and 0.75″, or more specifically between 0.125″ and 0.250″. For a simple round terminal feature 3, the diameter would typically be larger that the diameter of the post feature 2 in order to prevent removal from the piercing. The maximal diameter of the terminal feature 3 can range between 0.005″-2.000″, or more specifically between 0.050″-0.250″. Additional terminal feature 3 designs can be comprised of a simple cross bar (feature) or merely a sharp bend or pronounced curve in the post feature 2 (not shown), so as to limit the removal of the implant. The thickness of the terminal feature 3 can range between 0.001″ and 0.500″, or more specifically between 0.020″ and 0.125″.

The thickness of the bioabsorbable feature 5 can range between 0.0001″ to 0.250″, or more specifically between 0.010″ and 0.025″. Thickness of the bioabsorbable feature may also vary based upon materials used and application properties. Furthermore, it should be understood that the thickness of the bioabsorbable feature 5 can be varied during the deposition process in order to customize the different elution rates and durations at selected regions of the device.

The implant device can be made from any suitable material, for example metal, such as any alloy of stainless steel, gold, silver, titanium, platinum, cobalt-chromium, plated metals, or any plastic, for example polyethylene, polypropylene, polycarbonate, polyethylethylketone (PEEK), polyethylketoneketone (PEKK), high-density-polyethylene (HDPE), low-density polyethylene (LDPE), or ceramics, for example, aluminum oxide (alumina), zirconium, sapphire, etc. The implant device can be fabricated using manufacturing techniques known in the art, for example, conventional machining, CAD machining, casting, sintering, lost-wax casting, silver-soldering, laser-welding, electric discharge machining (EDM), grinding, bending or forming, injection molded, selective laser sintering (SLS), stereo lithography (SLA), gas-welding, resistance welding, or tungsten-inert-gas (TIG) welding.

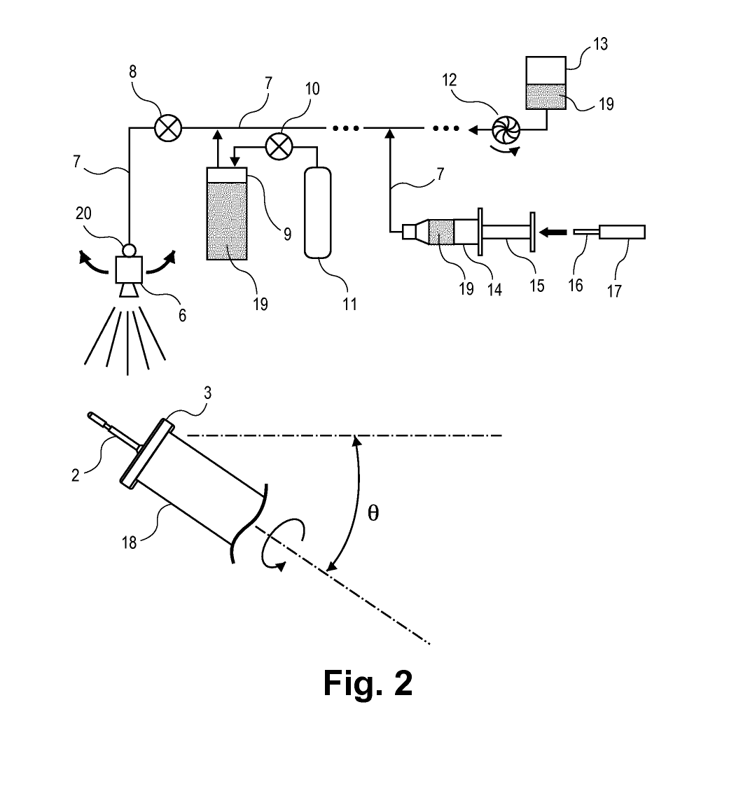

Various techniques can be employed to apply, coat, deposit, and assemble an implant device with bioabsorbable coating 1. The bioabsorbable coating(s), cover or sleeve can be applied either by spraying, dipping, casting, over-molding, or by attaching a prefabricated sleeve over the pin or post feature. In some embodiments, the technique employs an apparatus for use with a supply of bioabsorbable material to prepare the implantable device. The apparatus may include a framework having one or more supports thereon for support of the implantable device during the process and for support of or for engaging the application mechanism or supply of the bioabsorbable medium. One technique for creating the bioabsorbable feature 5 or applying the bioabsorbable feature, as depicted in FIG. 2, can include mounting the terminal feature 3 of the implant onto a support, such as rotating shaft 18 of an apparatus, such that the post feature 2 is mostly axially aligned with the rotating shaft 18, where the terminal feature 3 is held to the rotating shaft 18 by an adhesive backing, a simple clip or clamp, an adjustable fixture like a drill chuck, or by boss features in a receptacle that have an interference fit (not shown). To this end, the support may temporarily retain the implantable device thereon. The rotating shaft 18 can be supported by bushings, bearings, or the like.

An application device may be used to apply bioabsorbable material to or contact bioabsorbable material with the implantable device. While specific application devices or apparatus are described herein, any device or equivalents suitable for the purposes provided would be acceptable for use in applying the bioabsorbable material to the implantable device. To this end, a second support carried by the framework and adapted for engaging the supply of bioabsorbable material may be provided. The second support is preferably positioned relative to the first support so that the bioabsorbable material in the supply carried by the second support contacts the implantable device carried by the first support. In one embodiment, a spray nozzle 6 is provided for spray of bioabsorbable material onto the implantable device. A spray nozzle 6 that is purposely designed to accommodate the liquid bioabsorbable medium 19 and to create the desired spray pattern, as understood by persons skilled in the art, is mounted at the end of tubing 7, made from either metal or plastic, and is controlled via a nozzle valve 8, which can be manually adjusted or electronically controlled, either by a electric motor or a solenoid (not shown). The spray nozzle 6 can have any suitable spray pattern, for example a flat, cone, hollow cone, or square spray pattern. If employed in a larger automated process, more than one spray nozzle 6 can be assembled in an array (not shown) that could be used to cover a larger area. The angle of the spray nozzle 6 may also be adjusted about a pivot 20 during the spray process, either manually or by an automated mechanism (not shown). The spray nozzle 6 position may also be controlled along a single linear axis or multiple linear axis by mounting the spray nozzle 6 on one or more linear slides, for example, a re-circulating ball pillow block that translates on a shaft, a bushing that translates on a shaft, and the like. A pressurized gas cylinder 11, which can be simultaneously filled via an attached compressor (not shown) to maintain pressure, provides pressurized gas to a holding tank 9 by means of a tank valve 10 that can also be fitted with a pressure regulator (not shown) to accurately maintain the pressure within the holding tank 9.

The holding tank 9, containing liquid bioabsorbable medium 19, is connected to the nozzle valve 8 with a tubing 7, similar to as suggested before. The flow through the nozzle valve 8 can be controlled simply by turning the valve on and off manually, or in a timed manner, for example with an electronic timing device given a calculated flow rate determined by the spray nozzle 6 characteristics and the pressure in the holding tank 9. Additionally, the nozzle valve 8 can be controlled electronically, if, for example, the valve is actuated by a solenoid or a motor by utilizing a microprocessor, microcomputer, microcontroller or any other electrical or electronic control device to turn on and off the valve. The valve may, for example, be controlled with a variety of control schemes, such as, but not limited to, simple timed on-off, pulse-width-modulation (PWM), or proportional control in the case of a motor controlled proportioning type valve.

Additional mechanisms for pressurizing the liquid bioabsorbable medium 19 are also suggested in FIG. 2. A pump 12, for example, a rotary vane, a diaphragm, a peristaltic, or any other style can be utilized to supply pressurized liquid bioabsorbable medium 19 from the holding tank 13 through the nozzle valve 8 to the spray nozzle 6. Additionally, a piston-type plunger mechanism that includes a barrel 14 and a plunger 15, for example a plastic syringe, could be driven by an pushrod 16 in order to pressurize the liquid bioabsorbable medium 19 housed within the barrel 14. The pushrod 16 can be affixed to an actuator 17 which may include a pneumatic cylinder, an electric solenoid device, or a motor-driven linear actuator with a ball-screw or threaded drive, or any other similarly controllable displacement device. The actuator 17 can be controlled by a microcontroller or microprocessor to provide calculated displacement at programmed intervals in order to create a suitable amount of pressure at the spray nozzle 6 to create the desired volume and pattern of sprayed liquid.

The otologic implant which includes the post feature 2 and the terminal feature 3 may be oriented anywhere from 0 to 90 degrees relative to the horizontal axis, as shown in FIG. 2 and may change while the spray is applied or remain at a fixed angle. Alternatively, the spray can be applied at angled intervals to provide coatings of various thicknesses by preferentially applying more material to one region or the other. Additionally, the rotating shaft 18 may remain fixed during a spray cycle or may be indexed to various angle locations or may continuously rotate while spraying to provide either non-uniform or uniform distribution of the spray pattern.

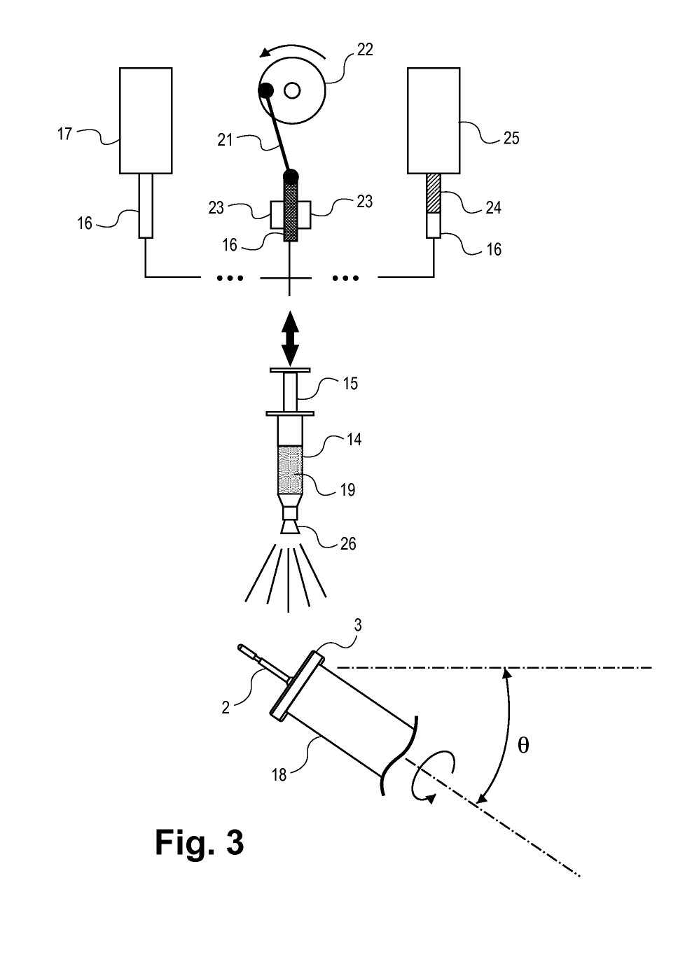

As an alternative embodiment of the pressurized liquid spray system depicted previously, a barrel 14 of a plastic, metal, or glass containing liquid bioabsorbable medium 19, as shown in FIG. 3, can be fitted with a mountable spray nozzle 26, either pre-assembled by a manufacturer or fitted to the tip of the syringe by the end-user. The mountable spray nozzle 26 could have a standard luer-type attachment that would interlock and seal with a typical syringe tip. Alternatively, the specific design features of the mountable spray nozzle 26 could be molded directly into the barrel 14, as an integral part of the barrel 14, to simplify the components, eliminate leaks, and to eliminate excess parts. The plunger 15 is then advanced into the barrel 14 by a pushrod 16 from an actuator 17, for example an electric solenoid or pneumatic cylinder. In addition, a crank 22 and connecting rod 21 fitted to a pushrod 16 constrained between shaft supports 23 provides a similar linear motion from a rotary input that can be provided by a motor or rotary solenoid (not shown). A similar linear motion also can be achieved from a linear drive 25 mechanism that uses a motor to rotate a ball-screw or threaded shaft 24 connected to a pushrod 16. As mentioned before, the actuators and drive systems can be interfaced to a microcontroller or microcomputer for precise control of the deposition layer from the spray. Similar to the previous embodiment depicted in FIG. 2, the implant can be held in a rotating shaft 18 and positioned at an fixed or variable angle for deposition of the sprayed bioabsorbable medium.

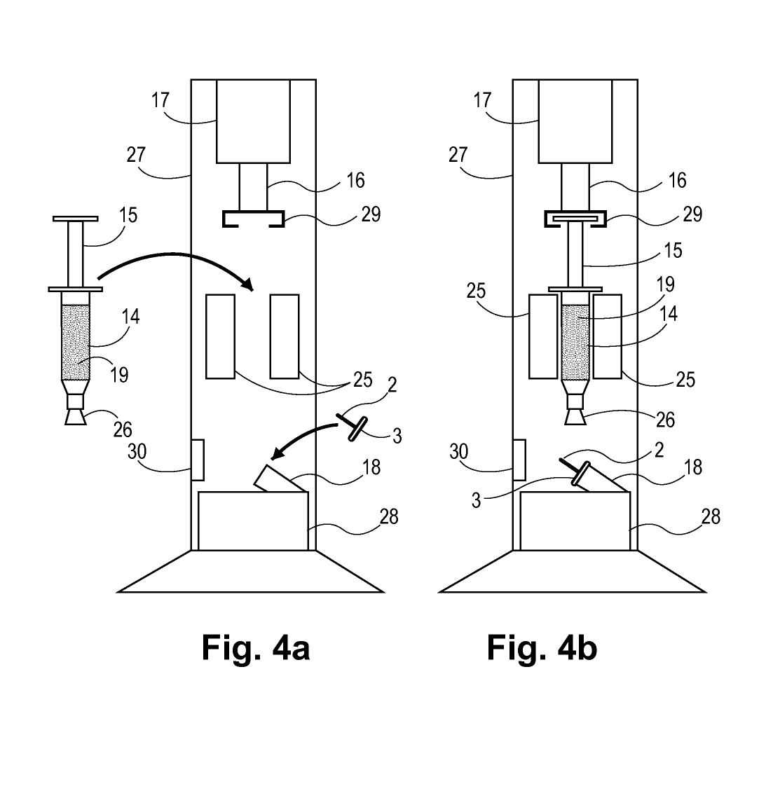

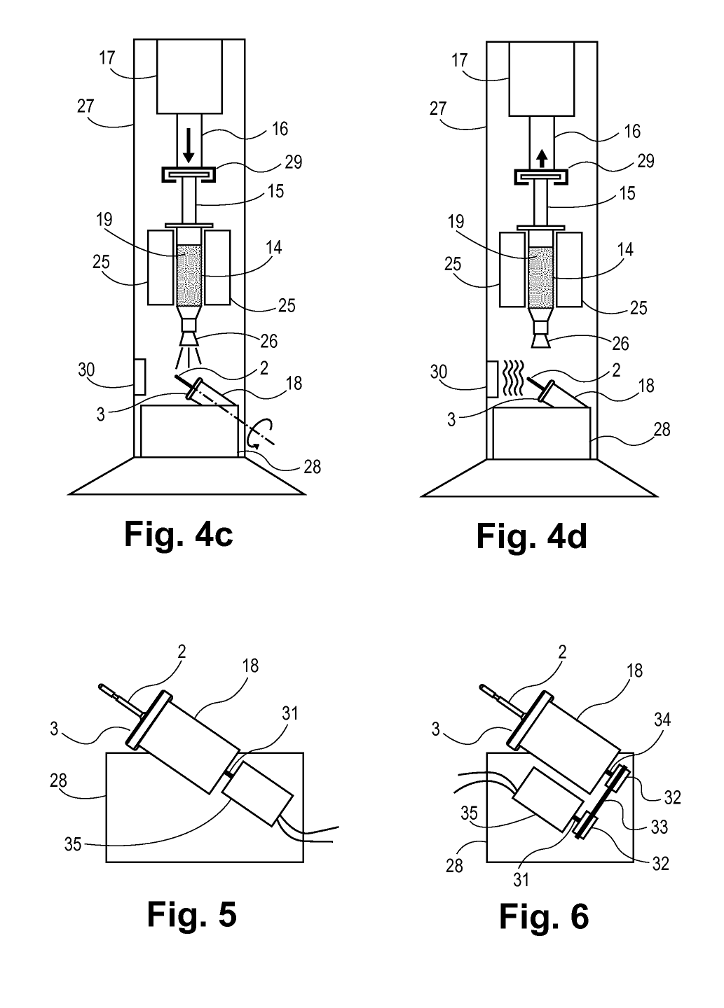

Utilizing the aforementioned concepts, a stand-alone system, like the device depicted in FIG. 4a, can be devised to include all the necessary mechanisms to automate the spray deposition process for an end-user. The rotating shaft 18 can be fitted to a shaft housing 28 that contains a drive mechanism (not shown) which can control the position, speed, and rotation direction of the rotating shaft 18. The actuator 17 can be mounted to a vertical support member 27. The pushrod 16 can have a capture feature 29 that can interlock with the plunger 15 end of a syringe, so that pushrod 16 can both advance and retract the plunger 15. A heater 30 or other drying mechanism can also be positioned along the length of the vertical support member 27 to aid in the drying of the spray coated layers. An implant can be mounted within the spray system onto a rotating shaft 18, as described previously, and a syringe-type vessel, for example, that is composed of a barrel 14 containing liquid bioabsorbable medium 19, a plunger 15, and a mountable spray nozzle 26, can be affixed within support features 25 and the plunger 15 interlocks with the capture feature 29, as shown in FIG. 4b.

The spray process begins, as shown in FIG. 4c, by turning on the drive mechanism to spin, index, or position the rotating shaft 18. The actuator 17, which can be any of the previously mentioned mechanisms, is activated to drive the pushrod 16 against the plunger 15, which is interlocked by the capture feature 29. Bioabsorbable liquid medium 19 contained in the barrel 14 is pressurized and then discharged in the spray pattern, as determined by the mountable spray nozzle 26, onto the implant, which consists of the post feature 2 and the terminal feature 3. The actuator 17 can then be commanded to retract the pushrod 16 slightly, as shown in FIG. 4d, such that the initial positive pressure in the barrel 14 imparted during the spray process is reduced to zero, or even to create a slight vacuum, which can help to minimize the amount of excess liquid that may continue to leak, drip, weep, or ooze from the mountable spray nozzle 26. This technique is commonly used in industrial fluid dispensing systems to better control the flow of the dispensed fluid and to limit the amount of wasted fluid lost to drips. A heater 30 can then be used to reduce the drying times after the spray has been deposited onto the implant and to possibly improve the speed at which layers of bioabsorbable liquid medium 19 can be placed on the implant and to build thin, but uniform layers. Alternatively, thick layers may also be deposited more effectively by quickly heating and solidifying sprayed-on layers that would normally tend to not remain in place if allowed to solidify at more ambient temperatures. Furthermore, by controlling the rotation of the implant, localizing the sprayed layer to certain localized regions, and then heating to solidify the layer provides for application in non-uniform thicknesses providing coatings that have regions of more and less drug elution. Also repeated deposition of layers may be used on specific portions of the implant for additional buildup of the coating in that particular region.

Methods other than heat can be used to solidify the deposited material, for example, exposure to ultra-violet light, blow with dried compressed air (heated or otherwise), or spray with a chemical activator. Commonly known heating mechanisms may also be used to dry the material applied to the implant. Alternatively, coating(s), cover or sleeve can be formed, cast, or sprayed onto an otologic implant by combining polysaccharide precursor reagents and allowing them to harden, as is understood by people skilled in the art.

Drive mechanisms for rotating the implant may include, but are not limited to, a direct-drive type design where the motor shaft 31 of the motor 35 can be connected to the rotary shaft 18 that in turn is fitted to the terminal feature 3 of the implant, as shown in FIG. 5. A pulley 32 mounted to the motor shaft 31 of the motor 35 and a pulley 32 mounted to the rotary shaft 18 via a shaft 34 that are interconnected with a belt 33 allows for a more compact and flexible packaging in the shaft housing 28. The aforementioned belt drive mechanism also allows for the speed ratio between the rotary shaft 18 and the motor 30 to be defined by the relative diameters of the each pulley 32. Given a fixed motor speed, the rotary shaft 18 speed can be specified to provide the desired rotation rate for the deposition process, which can range from 0-1000 revolutions per minute (RPM), or more specifically, between 1 and 10 RPM. The belt 33 can be a toothed-belt, v-belt, flat belt, round belt or any other type of belt design. A similar mechanism can also be devised with gears that mesh together or with rubber wheel(s) placed in intimate contact with another rotating element, such as directly to the motor shaft or another plastic or rubber wheel. The motor 30 can be a direct-current (DC) motor, and alternating-current (AC) motor, a servo motor, a stepper motor, an air motor, or spring motor. The motor 30 or drive mechanism within the shaft housing 28 may also have a means of detecting a rotational position by means of a switch or switches, a potentiometer, an optical encoder, or electric contacts, in order to repeatably locate a position or orientation of the implant during the deposition process. In addition, the implant can be rotated about its central axis with the mechanism described above, as well as pivoted about the angle θ, as shown in FIG. 2, with similar types of motor drives and crank mechanisms.

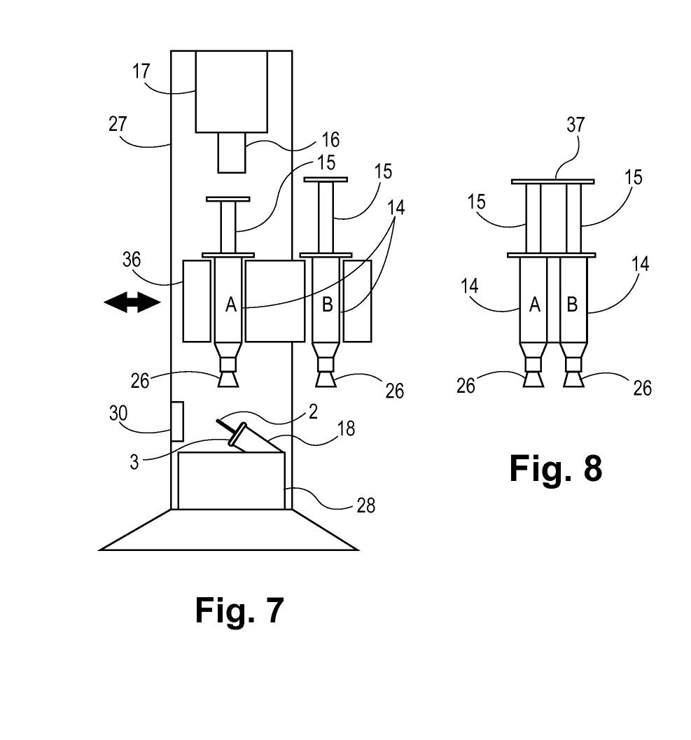

In the event that two different bioabsorbable liquid mediums or more than one or a plurality of bioabsorbable mediums are needed for covering the implant, the previously described spray mechanism can be designed to accommodate two individual barrels 14, as depicted in FIG. 7, each fitted with a plunger 15. A slidable holder 36 can be controlled to position either the barrel 14 containing component ‘A’ or the barrel 14 containing component ‘B’ to be axial with the pushrod 16 of the actuator 17. The slidable holder 36 can be mounted on bushings or on a linear track and can be controlled with a stepper motor, servo motor, or solenoid (not shown) or any other suitable mechanism.

A system that can dispense from two barrels 14, as shown in FIG. 7, can be used to apply bioabsorbable liquid medium 19 with two different medicaments or the same medicament with two different elution rates. Additionally, this system could be used to dispense bioabsorbable liquid medium 19 from one barrel 14 than contains medicament and the other barrel 14 that contains no medicament in order to create coatings with and without medicament. By varying the layers that contain medicament with ones that do not contain medicament allows for periodic or timed release of the medicament by forming a dissolvable barrier that then exposes the layer with medicament. The slidable holder 36 can accommodate two separate barrels 14 and can be dispensed independently with specific amounts delivered from each barrel. It is understood by those in the art that by simple modification the mechanism can accommodate additional barrels 14. In the case of a two-component material that requires even amounts of each material to be combined for proper hardening, a syringe-type device with two barrels 14, each driven by plungers 15 that are interconnected by a bridge 37 could be used, as is shown in FIG. 8.

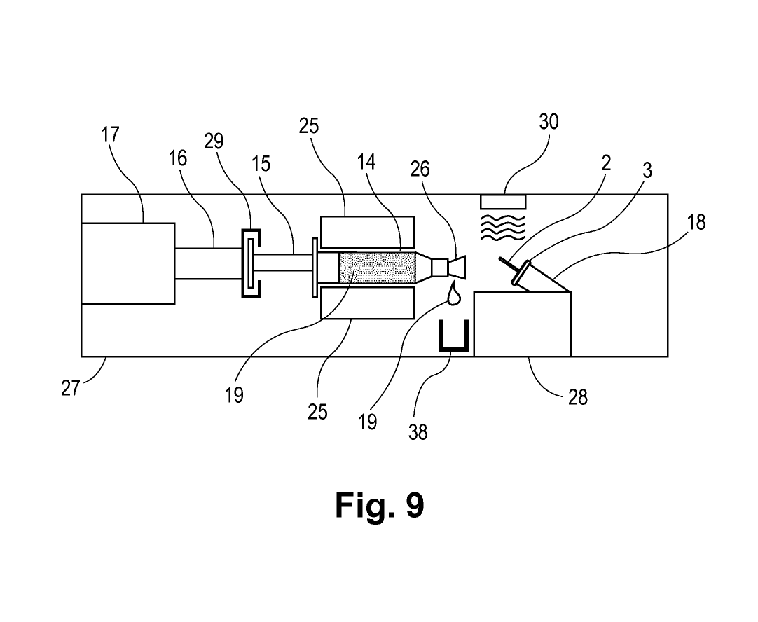

To prevent drips, leakage, oozing, or residual amounts of liquid bioabsorbable medium 19 from the mountable spray nozzle 26 inadvertently contacting the implant surface, the whole system can be mounted horizontally such that any leaks from the mountable spray nozzle 26 would be collected in a trap 38, as depicted in FIG. 9.

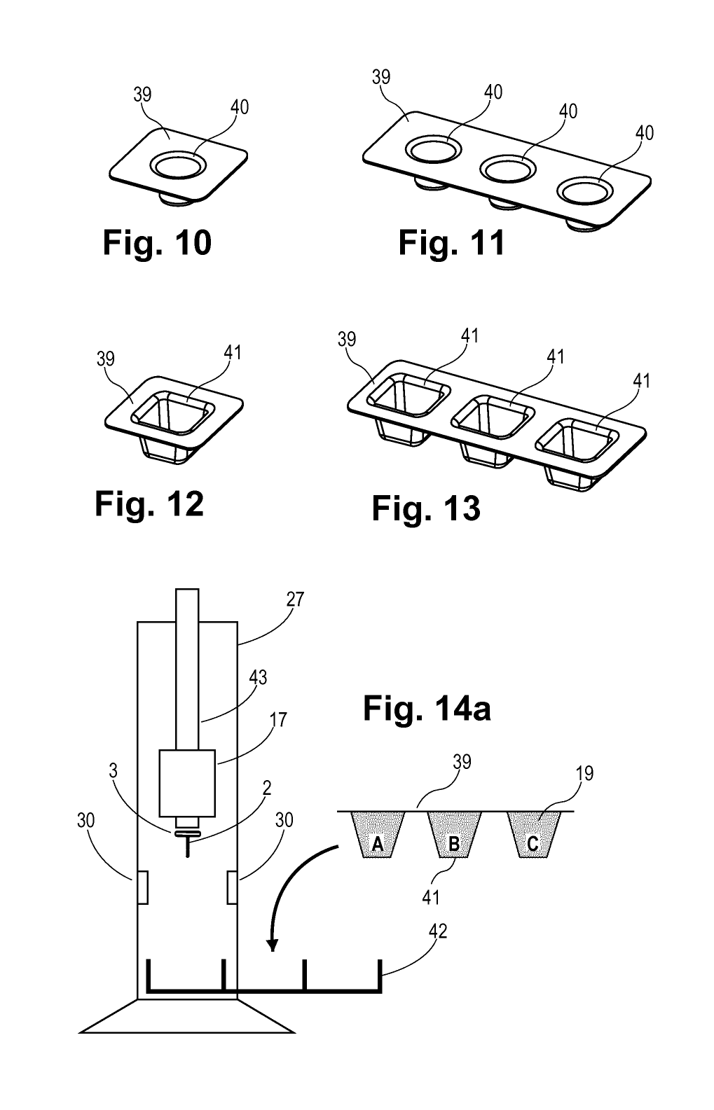

Dipping can also provide a suitable coating. To this end, the implant may be moved between a first position in which it is out of contact with a bioabsorbable medium, and a second position in which it is in contact with a bioabsorbable medium. Dipping the otologic implant, by use of a device or by hand, into a tray 39 with a receptacle 40 that contains liquid bioabsorbable medium 19 can thus be used to provide an external coating. A tray 39 with a single receptacle 40, or round receptacle, containing liquid bioabsorbable medium 19 (not visible), as shown in FIG. 10, can be packaged with a tear-away lid (not shown) and be stored until the time of use. Once the tear-away lid is removed, the user can simply dip the implant by-hand into the receptacle (not shown). The user can then let the coating dry in ambient air or apply heat, or a combination thereof, or use any other source to the implant to aid in the drying, polymerization, or solidification of the coating. Additional coatings can be made by re-dipping and then allowing that layer to solidify as well. As before, the coating process can be repeated until the desired thickness is achieved. For multiple drugs or for a single drug with multiple concentrations for different elution rates or for mediums with and without drugs, as described previously, a tray 39 with multiple receptacles 40. such as round receptacles, can be used to create customized dipping layers in a single coating session, as depicted in FIG. 11. The rectangular receptacles 41 can also be used, as depicted in FIG. 12 and FIG. 13, as well as hexagonal, oval, or any other geometry that can accommodate an implant. The receptacles can be straight walled or tapered. A tray 39 can contain one or more than one receptacle. The trays 39 can be made from metal or plastic or a combination of the two. The receptacles 41 can be hydro-formed, stamped, die-cut, vacuum-formed, injection-molded, cast, assembled from individual components or made with any other conventional manufacturing methods.

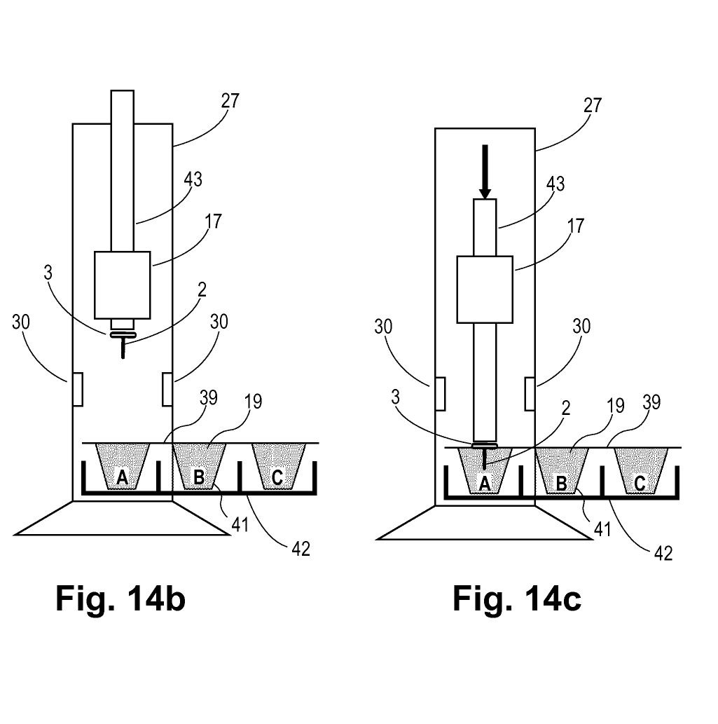

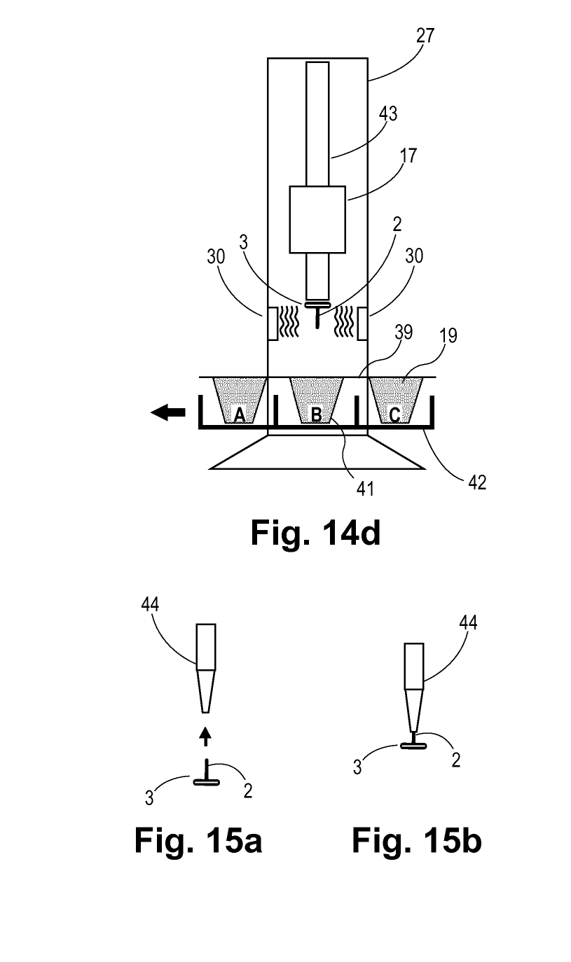

A mechanism for performing the dipping process is depicted in FIG. 14a, whereby an otologic implant is mounted to a dipping shaft 43 that would have similar attachment features as the previously described rotating shaft 18 in the spray-based methods. A tray 39 with receptacles 41 containing liquid bioabsorbable medium 19 is mounted within a carrier 42, as shown in FIG. 14a. An actuator 17, similar to the one described above, is used to raise and lower the otologic implant, composed of the post feature 2 and the terminal feature 3, in and out of the receptacle of the tray. Heater elements 30 may also be mounted to the vertical support member 27.

The carrier 43 can accommodate trays 39 with one or many receptacles 41, which can contain different formulations, concentrations, and compositions, as demarked by “A”, “B”, and “C” in FIG. 14b, for illustration purposes. The actuator 17 of the dipping mechanism can be advanced, using methods described previously, until the otologic implant, mounted at the end of the dipping shaft 43, is placed in the liquid bioabsorbable medium 19 “A”, as depicted in FIG. 14c, for any specified amount of time. The actuator 17 then retracts the otologic implant from the receptacle 41, as shown in FIG. 14d, and then locates the otologic implant in-between the heater elements 30.

Multiple coatings can be made in composition “A”, whereby the dipping and drying sequence can be repeated until the desired coating thickness is achieved, at which point, the carrier 42 can then be translated laterally by means of a stepper motor, solenoid actuator, rotary indexing mechanism, or any similar mechanism (not shown). The dipping sequence, as previously described, can then be repeated to create a coating of composition “B”. Similarly, the carrier 42 can be translated laterally again so that a coating of composition “C” can be made. Coatings with alternating compositions can be made by dipping in the desired sequence, for example, from the innermost to the outmost coating can be in the order: “A”, “B”,” “C”, “A”, “C”, “C”, “B”. This type of customization can be desirable to achieve a specific therapeutic effect. For instance, the outermost coating may initially be a thin, short elution time anesthetic coating to help mitigate the initial pain after the piercing procedure, the next innermost coating could be a thicker, longer elution-time anti-inflammatory layer to treat the subsequent swelling, and the final innermost layer could be a thicker, longer-elution antibiotic layer to minimize infection during the healing process. It should be understood that any sequence or number of layers containing various medicaments can be achieved using the above mentioned methods and the techniques.

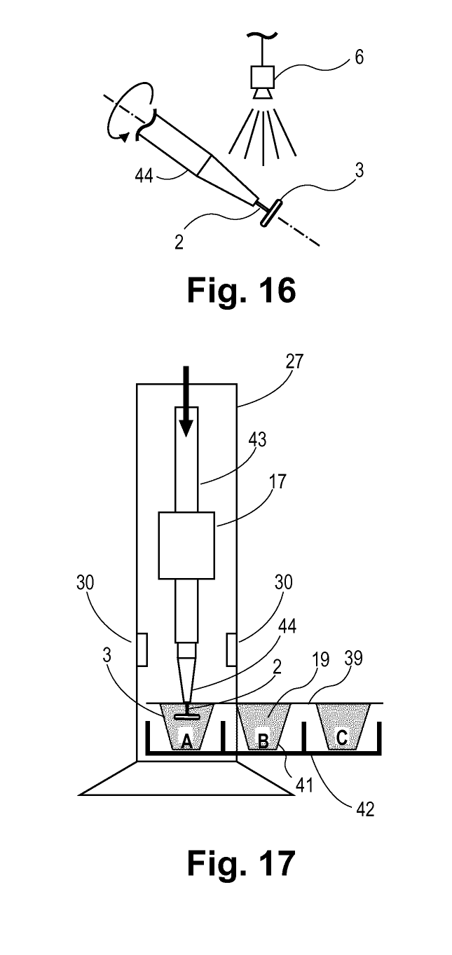

It should also be understood that the otologic implant can be held by the post feature 2 during the coating process, as well, using a holder 44 that has a hole at the tip (not shown) to accept the post feature 2, as shown in FIG. 15a and FIG. 15b. The hole (not shown) in the holder 44 can be a press-fit or can have a squeeze-type clamp mechanism or can be fitted with a set-screw (not shown) to accommodate slight variations in the diameter of the post feature 2. The holder 44 can also have a one or more thin slits long the length of the hole (not shown) that allow the tip feature to flex lightly to provide a slight compression fit and to also accommodate post features 2 with slightly varying diameters. The terminal end of the holder 44 that accepts the otologic implant can be tapered to minimize any interference with the spray pattern, as demonstrated in FIG. 16. The holder 44 can be made of any suitable plastic or metal. For example, a plastic holder 44 could have a slightly undersized hole to allow for a light press-fit that can be easily mounted by hand. The holder 44 and otologic implant assembly, depicted in FIG. 15b, can then be mounted, via a fixture or clamp, into similar coating devices, as previously disclosed, although the position may have to be slightly adjusted to accommodate the new holding orientation, as depicted in FIG. 16. The dipping device can accommodate the holder 44 with little to no modification, as seen in FIG. 17. Holder 44 may also accommodate more types of custom or customer-selected jewelry because it captures the otologic implant by the post feature 2 and not the terminal feature 3, which may be ornate in design and may be difficult to grasp with a universal clamping mechanism. By covering just the tip portion of the post feature 2 with the holder, the appearance of the final coated otologic implant is similar to that shown in FIGS. 1a, 1c, 1d, where there is no coating at the tip of the post feature 2 to interfere with the clasp 4.

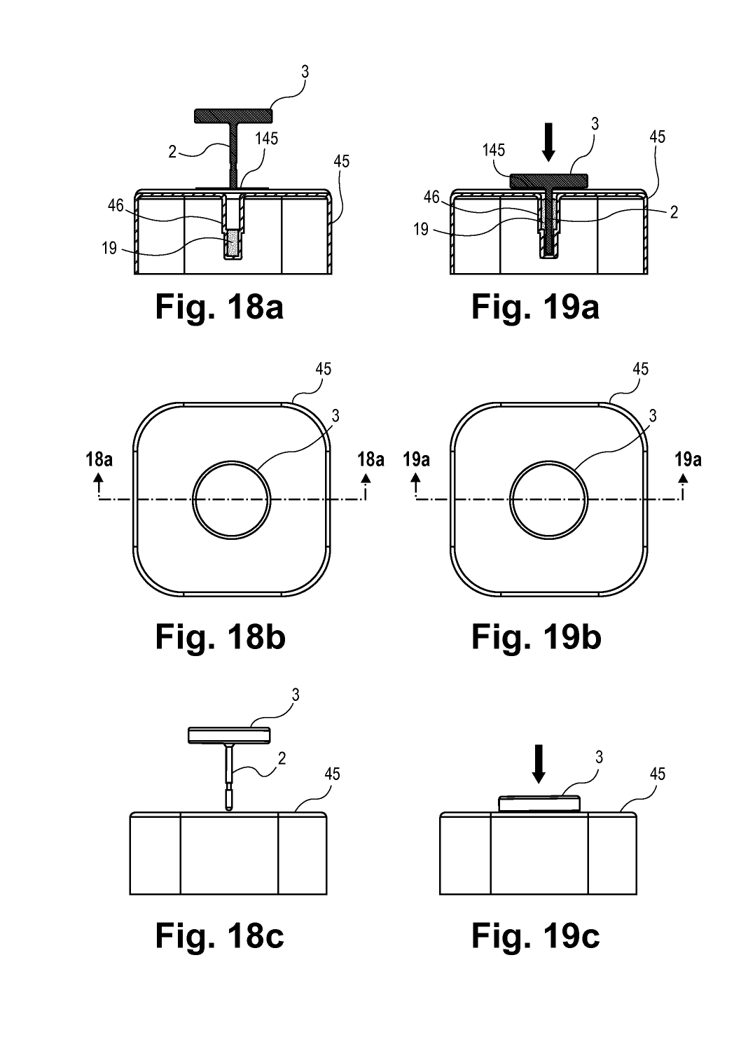

Another means of creating a coating on an otologic implant is to cast or over-mold the bioabsorbable coating directly onto the post feature 2 in the desired shape without multiple spray or dip coats. For example, a molding cup 45 can contain the liquid bioabsorbable medium 19 within a well 46 that has the desired final shape for the coating. The molding cup 45 can be vacuum formed from a polymer sheet, injection molded, cast, or machined, using skills and methods known in the art. A removable cap, cover, screw-top, or peel-away seal, may be provided to prevent the contents of the molding cup 45 from drying or spilling or otherwise becoming contaminated. As illustrated in FIGS. 18a and 19a, the molding cup of a preferred embodiment may include a film 145 which extends over the top surface of the molding cup and is aligned to cover the well 46. Preferably, the film 145 may be any suitable film or cover formed of any suitable material, and preferably is a thin plastic film capable of being punctured by the post 2 of an implant device 1. To this end, a molding cup 45 may be provided having a seal or film cover 145 retaining the bioabsorbable medium in the well 46. The film may be attached to the molding cup by any means known in the art, including but not limited to adhesive and the application of heat, or may be integrally formed with the molding cup 45.

In an exemplary embodiment of a method using the molding cup 45, the molding cup 45 may be removed from a package including the molding cup 45, and optionally the otologic implant. Following the removal from the package, the otologic implant is aligned with the well 46 of the molding cup 45, as shown in FIGS. 18a-18c. The post feature 2 of the otologic implant is pressed into the well 46 of the molding cup 45, as depicted in FIGS. 19a-19c. As the post feature 2 is pressed into the well 46, the post feature 2 punctures the film 145 if present. The post feature is further pressed into the well 46 such that the tip of the post feature 2 fits within the narrowest portion of the well 46 which preferably minimizes the amount of over-molding at this region. The majority of the liquid bioabsorbable medium 19 is displaced and allowed to collect around the post feature 2 closest to the terminal feature 3, where the over-molding is intended, as clearly seen in the cross-sectional view taken along the line 19a-19a of FIG. 19b. The liquid bioabsorbable medium 19 is allowed to dry, which as indicated herein could be accelerated by placing the molding cup 45 onto a heated surface (not shown), for instance, a warming plate or other heated device. Once sufficiently dried, the otologic implant can be removed from the molding cup 45 and have a final form resembling the otologic implant in FIG. 1a.

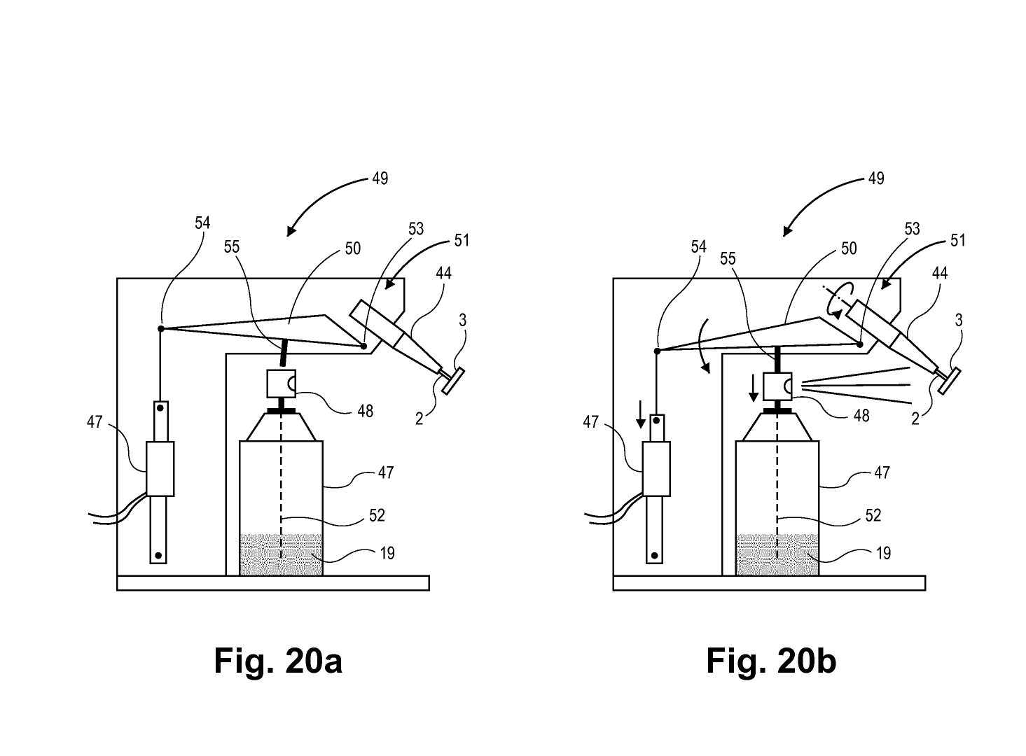

Another embodiment of a spray coating method is disclosed that uses liquid bioabsorbable medium 19 contained within a can 47 with pressurized gas (propellant) and a spray nozzle 48, similar to a conventional paint spray can, as shown in FIG. 20a. The can 47 is placed within a spray device 49 that has a pivoting lever 50 to actuate the spray nozzle 48 and a rotary mechanism 51, like the ones mentioned previously, for spinning the otologic implant. A lever actuator 47, similar to the actuators previously mentioned, is mounted at one end by a pivot to the lower portion of the spray device 49 and the other end to the free-end of the pivoting lever 54. By activating the lever actuator 47, the pivoting lever rotates about a hinge point 53, and in turn causes the nozzle boss 55 to press downwardly on the spray nozzle 48, which actuates a valve (similar to spray can), and then causes the pressurized liquid bioabsorbable medium 19 to exit the can through the spray nozzle 48, via a spray tube 52 within the can. The spray is focused on an otologic implant that is rotating about an axis, which like the previous embodiments, can actively pivot, given an additional mechanism (not shown). This configuration has the advantage of a portable pre-pressurized, easily disposable or recyclable can 47 using known methods in the art to manufacture the pressurized can, valve, and nozzle. The overall device is simplified, compared to the previously disclosed spray concepts, by eliminating the mechanisms required to pressurize the liquid contents. After the can 47 is emptied, it is discarded and a new (or different) can 47 is mounted within the spray device 49. Different cans 47 containing different medicaments or different concentrations of the same medicament can be used to create coatings on a single otologic implant. Furthermore, multiple spray devices 49 that are fitted with cans 47 containing different contents can be aligned to spray a coating on a single otologic device. A microcomputer or microcontroller can be used to control the individual spray cycles and sequence. Or even more simply, a user may want to manually spray an otologic implant while fixing it in a holder, between their fingers, or on a flat surface (not shown) and then depress the spray nozzle by hand, without any electric and electronic mechanisms, just like applying paint onto an object with a typical spray paint can.

Additionally, the devices, mechanisms, and methods disclosed can be utilized to apply a bioabsorbable coating(s), cover or sleeve to a broad range of earring pins or posts, therefore making the method suitable to accommodate custom jewelry. Furthermore, jewelry can be provided by customers, clients, or companies, either at the time of the piercing or as a service whereby individual or batches of many earrings are prepared and packaged. Implantable devices may also be prepared in bulk quantities and sold to individuals, stores, or companies that provide a piercing service.

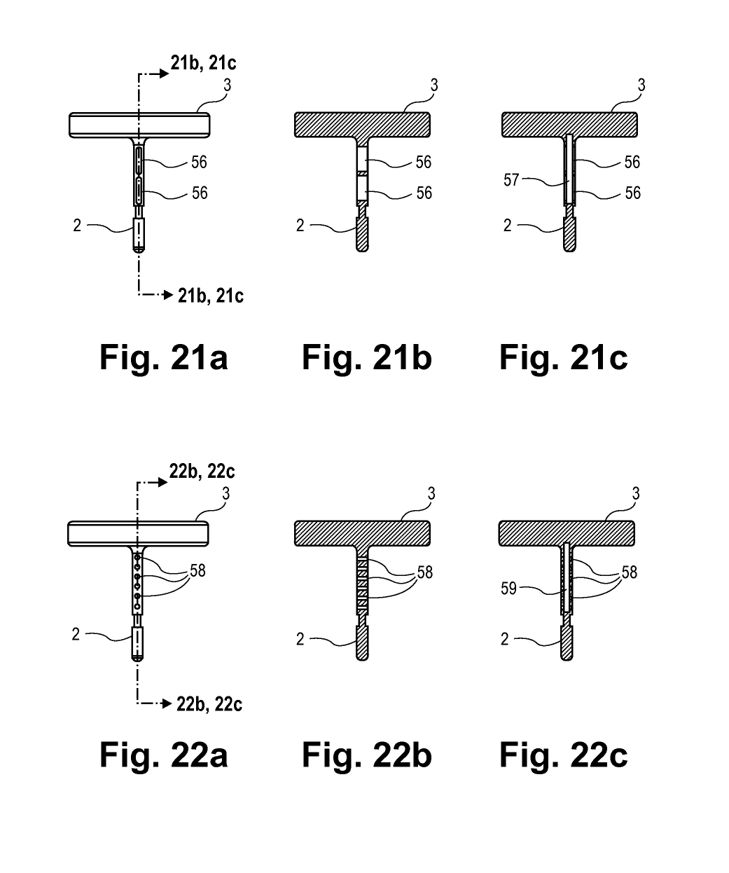

Although a simple round and smooth post feature 2 from a typical earring may be sufficient for most sprays, coatings, and castings, it may be preferred to have a post feature 2 that has a full-thickness slit 56 or series of slits along the length of the post feature 2, like those shown in FIGS. 21a-21b or the slit(s) 56 can in turn expose a hollow core 57 in the post feature 2, like that in FIG. 21c. These embodiments could potentially accommodate more volume of bioabsorbable medium during the spray, coating, and or casting process and could allow for even longer elution times or allow for larger concentrations of eluted medicament. The slit 56 width could range from 0.0001″ to 0.1″, or more preferably could range from 0.001 to 0.010″. The slit 56 lengths could range between 0.0001″ to 0.750″, or more preferably range from 0.010″ to 0.050″. An alternative embodiment has full thickness holes 58 instead of slits 56 in the post feature 2, as depicted in FIGS. 22a-22b, or holes 58 that also expose a hollow core 59 in the post feature 2, as shown in FIG. 22c. In addition to accommodating a larger volume of bioabsorbable medium, the holes 58 may have less of an effect on the mechanical integrity of the post feature 2 than slits 56 could have. The hole 58 diameter could range from 0.0001″ to 0.1″, or more preferably could range from 0.001 to 0.010″.

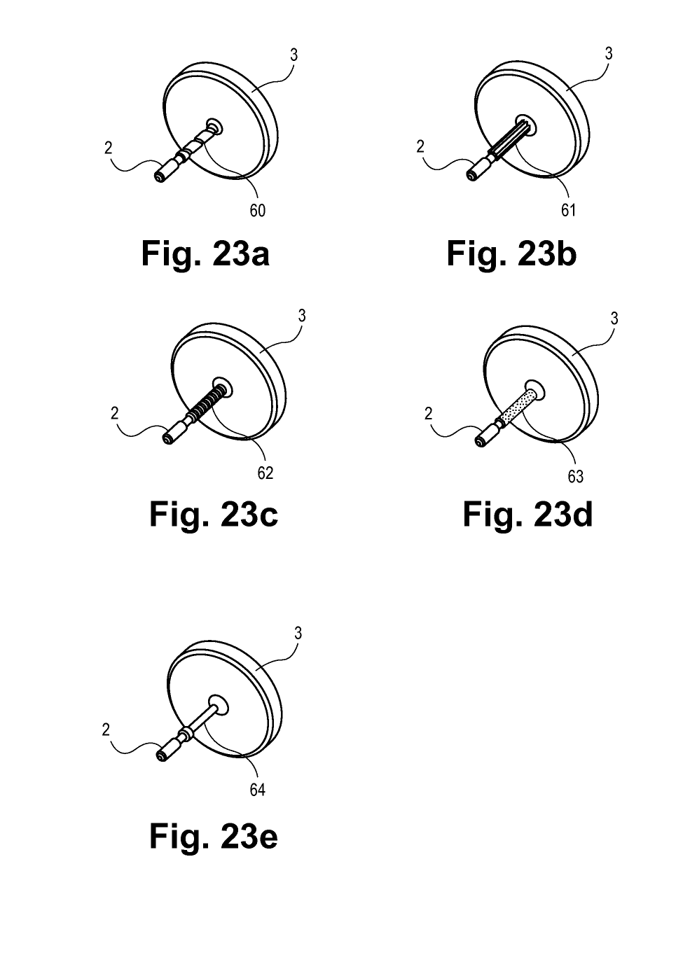

Additional embodiments of post features 2 of the otologic implant that can accommodate more volume of bioabsorbable coating or help stabilize the coating, or a combination thereof, are disclosed, including: a helical groove 60 or notch along the length of the post feature 2, as shown in FIG. 23a, longitudinal ribs or ridges 61 along the length of the post feature 2, as shown in FIG. 23b, circumferential ribs or ridges 62 along the length of the post feature 2, as shown in FIG. 23c, or dimples, pockets, or scallops 63, or a combination thereof, on the surface of the post feature 2, as depicted in FIG. 23d. More than one helical groove or notch, as depicted in FIG. 23a, can be incorporated into the post feature 2. Furthermore, an otologic implant with an undercut 64 or reduced diameter section along the length of the post feature 2 may be used.

These aforementioned surface features, especially the helical groove 60, may help vary the elution rate of the bioabsorbable medium over the course of the healing process. The initial coating of bioabsorbable medium that covers the entire post feature 2 may tend to elute faster, especially with the entire surface area of the coating in intimate contact with the pierced tissue in the ear. This configuration may be beneficial because the pierced tissue initially gets a larger dose of medicament. However, as that initial coating layer is absorbed, the surface area of the bioabsorbable coating exposed to tissue in the piercing is reduced to the regions exposed within the groove, ribs or ridges. This may ultimately reduce the elution rate and provide a more prolonged, yet lower dose of medicament, which could be more preferred during the final stages of healing. In addition, the helical groove 60 also has the added benefit that it can be relatively deep and still maintain sufficient mechanical integrity of the post feature 2. The undercut 64 feature depicted in FIG. 23e can be particularly useful when the coating is cast onto the post feature 2, as depicted in FIGS. 18a-18c and FIGS. 19a-19c. The well 46 can be configured such that the coating is flush with the rest of the post feature 2 and therefore would leave little or no transition between the coating diameter and the maximal diameter of the post feature 2. This may aid with the ease of insertion of the post feature 2 into the pierced tissue. These surface features can be utilized individually or combined in various configurations to tailor the elution process.

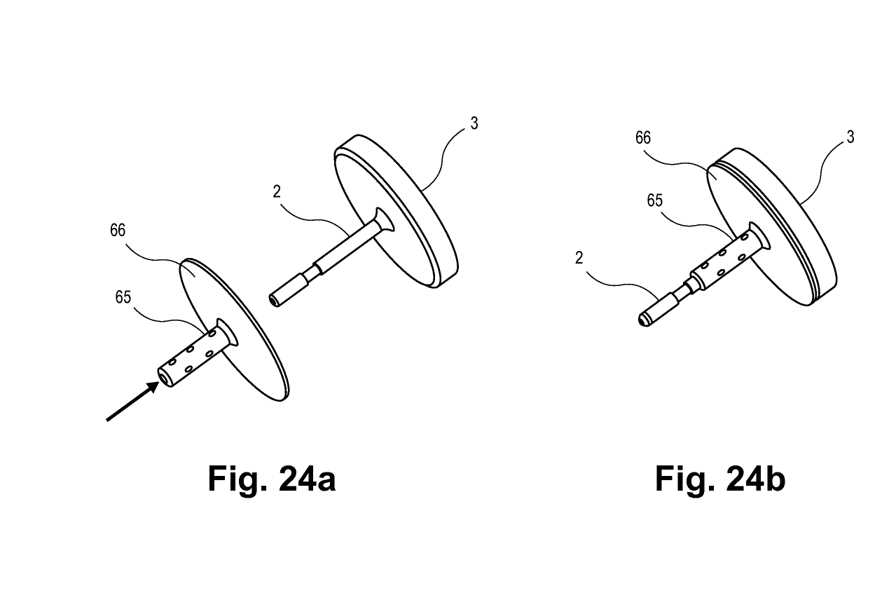

An additional embodiment includes a pre-fabricated sleeve 65, as depicted in FIG. 24a, that is cast, injection molded, or machined, or a combination thereof, and can be mounted directly onto a post feature 2 of an otologic implant. The sleeve 65 can have holes, scallops, or other surface modifications to increase the outer surface area for higher initial elution rates. The inner diameter of the sleeve 65 can be slightly smaller than the outside diameter of the post feature 2 of the otologic implant to create a press fit. The sleeve 65 can be continuous with a flange feature 66 that covers either a portion or the entire terminal feature 3 of an otologic implant, although the flange is not required. The sleeve 65 can be applied by hand or with the aid of a simple applicator that houses the sleeve (not shown). The composition of the bioabsorbable medium used for the sleeve 65 can be made preferentially softer, e.g. similar in consistency to a firm rubber, to allow for the sleeve 65 to stretch over the post feature 2. Tabs, hooks, arms, or ribs (not shown) can extend from the flange feature 66 in order to capture the terminal feature 3 of the otologic implant to provide additional fixation. The sleeve 65 can be pre-mounted to an otologic implant and packaged in a kit for use by an individual. The sleeve 65 can also be packaged separately in a kit and the individual can then mount the sleeve 65 onto the post feature 2 of an earring for a client or customer. The earring can be one supplied separately in a kit or the earring can be commercially made and purchased from the vendor or the earring can be supplied by the customer. The benefit of all the aforementioned embodiments is that it allows the customer to use an earring that they already own and may have sentimental value or is a design or configuration they prefer.

Using the foregoing devices and apparatus, a method of delivering a medicament to an externally-accessible pierced opening in a mammalian body is provided. The method generally includes applying a bioabsorbable material and a medicament to an implantable device, implanting the implantable device into the externally-accessible pierced opening, and eluting the medicament. As discussed, the implantable device may be a body piercing implant or may be an otologic implant. In one embodiment, the bioabsorbable material and medicament is applied by spray coating the implantable device. Alternatively, the bioabsorbable material and medicament are applied by dipping of the implantable device into the material and medicament. The foregoing application methods may further require the use of a drying mechanism or period of time to allow the material to dry on the implant. A further alternative may include applying or attaching a sleeve to the implantable device. While specific methods and steps are described, variations thereon, variations on the order of respective steps and equivalent or alternative devices would not depart from the overall scope of the present invention.

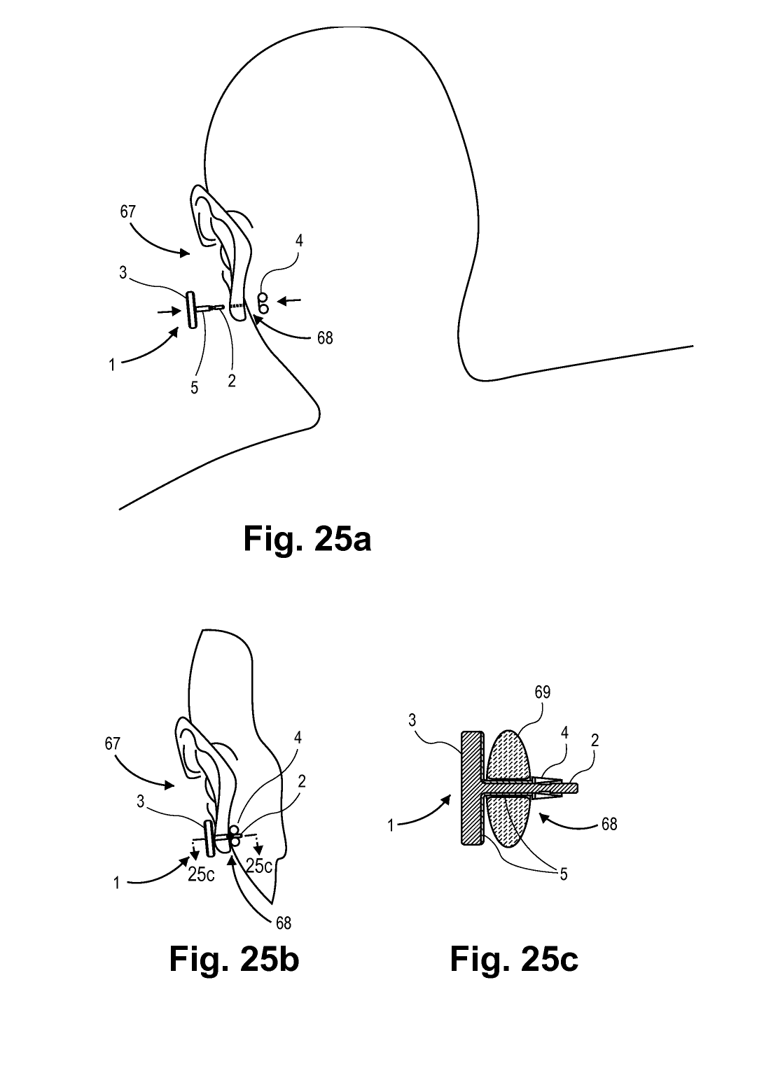

Once the bioabsorbable feature 5 is applied to the implantable device, it is inserted into the mammalian body. In one exemplary embodiment, an otologic implant with a bioabsorbable coating 1, consisting of a terminal feature 3, post feature 2, a bioabsorbable feature 5, and a clasp element 4, is shown in FIG. 25a being placed into a human lobe piercing 68 of the left ear 67. The post feature 2 is inserted into the externally-accessible pierced opening. The clasp element 4 is then securely fixed to the post feature 2 of the otologic implant with bioabsorbable feature 1 to capture the tissue of the lobe piercing 68, as depicted in FIG. 25b. A sectional view of line 25c-25c, illustrated in FIG. 25c, shows the bioabsorbable feature 5 in intimate contact with the lobe tissue 69 of the lobe piercing 68. Then, while the otologic implant with bioabsorbable coating 1 is in place, the pharmaceutical agent or medicament is eluted from the implant to treat the area.

In addition to the foregoing, a kit may be provided including one or more of the hereinabove described elements. Preferably, the kit is formed of a package 101 or container carrying one or more components. For example, a kit may be assembled of provided that contains an otologic implant or more than one otologic implant with a pre-applied coating(s) or the components necessary to assemble an otologic implant, at the time of use, with a bioabsorbable coating(s) or sleeve for the purpose of eluting one or more pharmaceutical agents. Accordingly, in an embodiment of the kit, all or any one of the components described herein or needed to create a coated otologic implant could be assembled into a kit.

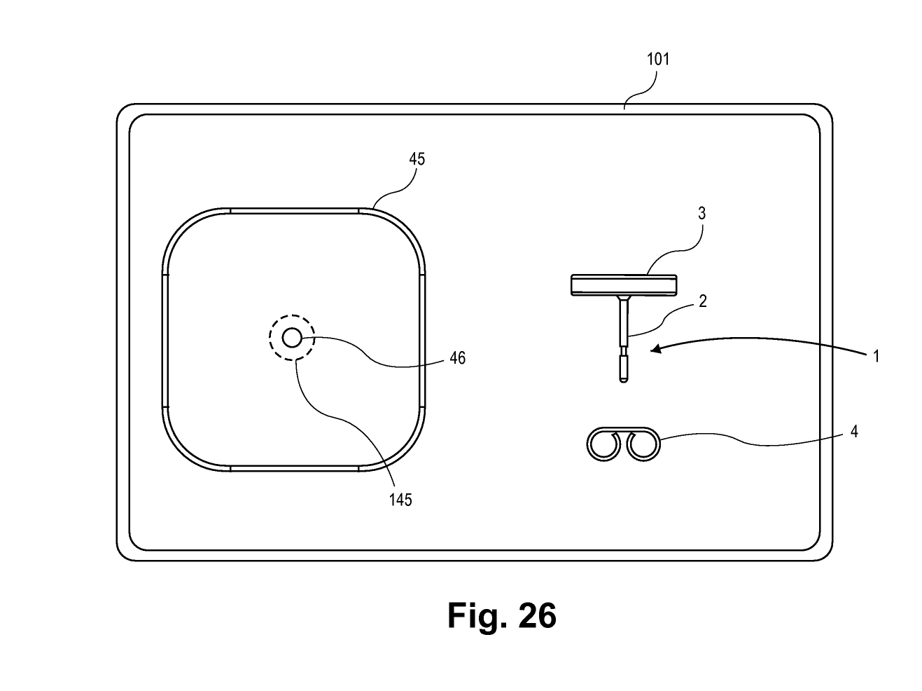

In an exemplary embodiment, as shown in FIG. 26, the kit may include a package 101 having a sealed molding cup 45 with liquid bioabsorbable medium contained in the well 46 (as shown in FIGS. 18a and 19a) and optionally one or more clean or sterilized otologic implants 1 designed to fit specifically into the well. A clasp 4 may also be optionally included in the kit. The reservoir in the exemplary embodiment of FIG. 26 is adapted for dipping of at least a portion of the implantable portion of the implant device therein. Alternatively, the kit may include a sleeve such as that shown in FIG. 24a, or be formed of a package containing a sleeve, which sleeve includes the bioabsorbable material and medicament. The sleeve is adapted to fit or be received by the elongate member, and more preferably the implantable portion of the elongate member.

While a molding cup is illustrated in the exemplary embodiment of FIG. 26, any application device or assembly or equivalent suitable for coating the implant may be substituted in place of the molding cup in the kit. For example, a stand-alone system or apparatus for automated spray deposition or a spray coating assembly as set forth in FIGS. 4a-4d or FIG. 9 may be included in a package of a kit, which package may also include a syringe, optionally pre-loaded with bioabsorbable medium. The package may further optionally include one or more implant devices 1 within in the container.

Similarly, a multiple-syringe spray coating assembly as set forth in FIGS. 7-8 may be provided in a package of a kit, and optionally may include one or more syringes or pre-loaded syringes preloaded with bioabsorbable medium. The package may further optionally include one or more implant devices 1 in the container.

Alternatively, a package or kit may be formed of a container including tray 39 of any one or more of FIGS. 10-13, and may optionally include one or more implant devices 1 within the container. These trays 39 may also be optionally pre-loaded with bioabsorbable medium in a sealed arrangement.

A dipping assembly or apparatus as set forth in FIGS. 14a-14d may, likewise be provided in a package or kit. The container of the kit may also optionally include one or more implant devices 1. This embodiment may also optionally include one or more trays 39 within the package.

An implant device holder 44, as shown in FIGS. 15a and 15b, may also be provided in a container or package, either as an optional component of a kit for a dipping assembly, or as a stand-alone kit including a holder 44 which may be adapted to various dipping assemblies or for use by hand. The kit containing the implant device holder 44 may also optionally include one or more implant devices 1 within the package. While holder 44 is specifically described, alternative implant device support mechanisms may alternatively be provided in a kit.

A further alternative embodiment of a kit includes a can 47 with pressurized gas and a spray nozzle 48 as shown in FIGS. 20a-20b. The can 47 may include the bioabsorbable medium. The can 47 may be provided, alone, in a package as a kit, or may further optionally include an assembly or apparatus 49 which mounts thee can 47 and implant device 1 in the kit. The kit may also optionally include one or more implant devices 1 within the package, and optionally include one or more implant device holders 44 within the package.

Any one of the foregoing described assemblies may be provided in separable components within a package or more than one package or may alternatively be provided in an assembled form. The implantable device in the foregoing described kits may also be substituted with any suitable implantable device described herein or equivalent. Furthermore, the implantable device or portions thereof may be included in the kit as integral components or distinct components to be combined into at least one implantable device. Alternatively, the package may not include the implant or may not include the application device for applying the bioabsorbable medium. The package may also separately include a container or reservoir of the bioabsorbable medium and pharmaceutical agent, individually or in combination. Thus, the package 101 or container may be or include a reservoir with a bioabsorbable material and pharmaceutical agent or medicament.

Even if not specifically described in the foregoing examples, it is contemplated that any one of the components may be an optional feature of the kit.

Accordingly, a kit formed of a package 101 having an implantable device for implanting into an externally-accessible pierced opening in a mammalian body is provided, which kit may also include a container having a bioabsorbable material and medicament for applying to the implantable device prior to insertion into the externally-accessible pierced opening.

While embodiments of the present invention have been shown and described, various modifications may be made without departing from the scope of the present invention. The invention, therefore, should not be limited, except to the following claims, and their equivalents.

I claim:

1. An implantable device for use in an externally-accessible pierced opening in a mammalian body comprising an implant having an elongated member adapted for insertion into the opening, a bioabsorbable material provided on at least a portion of the elongated member and a pharmaceutical agent carried by the bioabsorbable material for eluting into the mammalian body when the elongated member is disposed in the opening.

2. The implantable device of claim 1, wherein the bioabsorbable material is in the form of a coating.

3. The implantable device of claim 1, wherein the bioabsorbable material is in the form of a prefabricated sleeve.

4. The implantable device of claim 1, wherein the bioabsorbable material is selected from the group consisting of poly ester amide, polylactic acid, polyglycolic acid, and poly(lactide-co-glycolide)

5. The implantable device of claim 1, wherein the bioabsorbable material includes a plurality of bioabsorbable mediums.

6. The implantable device of claim 1, wherein the pharmaceutical agent is an analgesic.

7. The implantable device of claim 1, wherein the pharmaceutical agent is an anesthetic.

8. The implantable device of claim 1, wherein the pharmaceutical agent is an anti-inflammatory.

9. The implantable device of claim 1, wherein the pharmaceutical agent is a steroid.

10. The implantable device of claim 1, wherein the implant includes a terminal member for overlying the opening, the elongated member extending from the terminal member.

11. The implantable device of claim 1, further comprising a clasp element engageable with the implant for securing the implant in position on the mammalian body.

12. The implantable device of claim 1, wherein the elongated member has a surface feature for increasing the amount of bioabsorbable material provided on the elongated member.

13. The implantable device of claim 12, wherein the surface feature is selected from the group consisting of a slit, a groove, a rib, a ridge, a dimple, a pocket, a scallop, an opening, a bore and an area of reduced diameter.

14. The implantable device of claim 1, wherein the device is an otologic implant.

15. The implantable device of claim 1, wherein the implantable device is a body-piercing implant.

16. An otologic implant for placement in a pierced opening of an ear of a mammalian body comprising a terminal element adapted for overlying the pierced opening, a stud extending from the terminal element for placement in the pierced opening and having an outer surface, a layer of a bioabsorbable material provided on at least a portion of the outer surface of the stud and a pharmaceutical agent carried by the bioabsorbable material for eluting into the mammalian body when the stud is disposed in the pierced opening.

17. The otologic implant of claim 16 wherein the terminal element has front and back surfaces, the bioabsorbable material disposed on at least a portion of the back surface of the terminal element so as to overlie the pierced opening.

18. An apparatus for use with a supply of bioabsorbable material to prepare an implantable device for use in an externally-accessible pierced opening in a mammalian body comprising a support for temporarily securing to the implantable device, an application device for applying the bioabsorbable material to the implantable device, the application device being arranged to contact the implantable device secured by the support with the supply of bioabsorbable material and pharmaceutical agent and capable of applying the bioabsorbable material and pharmaceutical agent to the implantable device.

19. The apparatus of claim 18, wherein the support for an implantable device is adapted for rotation of the implantable device.

20. The apparatus of claim 18, wherein the support for an implantable device is adapted for movement of the implantable device between a first position and a second position.

21. The apparatus of claim 18, wherein the application device is a spray mechanism.

22. The apparatus of claim 18, wherein the application device is a receptacle for containing the supply of bioabsorbable medium and adapted to temporarily receive at least a portion of the implantable device.

23. The apparatus of claim 18, further comprising a drying mechanism for drying the bioabsorbable material applied to the implantable device.

24. An apparatus for use with a supply of bioabsorbable material to prepare an implantable device for use in an externally-accessible pierced opening in a mammalian body comprising a framework, a first support carried by the framework and adapted for temporarily securing to the implantable device, a second support carried by the framework and adapted for engaging the supply of bioabsorbable material, the second support being positioned relative to the first support so that the bioabsorbable material in the supply carried by the second support contacts the implantable device carried by the first support.

25. The apparatus of claim 24 wherein the first support is rotatably carried by the framework.

26. The apparatus of claim 24 further comprising a drying mechanism carried by the framework for drying the bioabsorbable material on the implantable device.

27. A kit comprising a package having an implantable device for implanting into an externally-accessible pierced opening in a mammalian body, the implantable device including an implantable portion, and a container having a bioabsorbable material and medicament for applying to the implantable device prior to insertion into the externally-accessible pierced opening.

28. The kit of claim 27, wherein the bioabsorbable material and medicament are carried by a sleeve adapted to be received by at least a portion of the implantable portion.

29. The kit of claim 27, wherein the bioabsorbable material and medicament are carried by a reservoir adapted for dipping of at least a portion of the implantable portion therein.

30. A kit comprising a package containing a sleeve carrying a bioabsorbable material and pharmaceutical agent, the sleeve being adapted for attachment to an implantable device for implanting into an externally accessible pierced opening in a mammalian body.

31. A method of delivering a medicament to an externally-accessible pierced opening in a mammalian body comprising applying a bioabsorbable material and a medicament to an implantable device, implanting the implantable device into the externally-accessible pierced opening, and eluting the medicament.

32. The method of claim 31, wherein the implantable device is an otologic implant.

33. The method of claim 31, wherein the implantable device is a body piercing implant.

34. The method of claim 31, wherein the applying step includes spray coating at least one of the bioabsorbable material and medicament.

35. The method of claim 31, wherein the applying step includes dipping at least a portion of the implantable device into at least one of the bioabsorbable material and the medicament.

36. A method for implanting a device having an elongated member provided with a free end in a pierced opening of a mammalian body comprising applying a bioabsorbable material to the at least a portion of the elongated member at the site of implantation, inserting the elongated member through the opening and securing a clasp element to the free end of the elongated member to retain the elongated member in the opening.

37. The method of claim 36 wherein the applying step includes spraying the bioabsorbable material on at least a portion of the elongated member.

38. The method of claim 36 wherein the applying step includes dipping the elongated member in the bioabsorbable material.

39. The method of claim 36 wherein the applying step includes placing a prefabricated form of the bioabsorbable material on the elongated member.

40. The method of claim 36 wherein the bioabsorbable material includes a medicament.
